(19) United States
(12) Patent Application Publication
(10) Pub. No.: US 2009/0054717 A1
(43) Pub. Date: Feb. 26, 2009

(54) Method of Producing 3,4-Diacyloxy-1-Butene

(75) Inventors: Tomohisa Okada, Osaka (JP); Yuji Yamaguchi, Osaka (JP)

Correspondence Address: GREENBLUM & BERNSTEIN, P.L.C., 1950 ROLAND CLARKE PLACE, RESTON, VA, 20191, US

(73) Assignee: THE NIPPON SYNTHETIC CHEMICAL INDUSTRY CO., LTD., Osaka (JP)

(21) Appl. No.: 12/194,031

(22) Filed: Aug. 19, 2008

(30) Foreign Application Priority Data
Aug. 21, 2007 (JP) 2007-214591

(51) Int. Cl. C07C 5/27

(52) U.S. Cl. 585/671

(57) ABSTRACT

This invention provides a method of producing 3,4-diacyloxy-1-butene by isomerization by heating 1,4-diacyloxy-2-butene in the presence of an isomerization catalyst, with a higher yield rate with respect to the supply amount of a starting material, even not changing a kind of the isomerization catalyst, a composition of the starting material, and/or a chemical structure of 1,4-diacyloxy-2-butene used as the starting material. This advantage is achieved by preventing the isomerization from terminating through suppressing attaining equilibrium.

The isomerization is conducted while distilling away 3,4-diacyloxy-1-butene (the isomerized product) with use of a reactor equipped with a distillation device. The isomerization is preferably carried out under heating to a temperature of not less than the boiling point of 3,4-diacyloxy-1-butene. Also, the isomerization is preferably carried out under a reducing pressure. Further preferably, 1,4-diacyloxy-2-butene is continuously supplied and 3,4-diacyloxy-1-butene is continuously distilled away during isomerization.

### BACKGROUND OF THE INVENTION

1. Field of the Invention

The present invention relates to a method of producing 3,4-diacyloxy-1-butene by isomerization of 1,4-diacyloxy-2-butene, and more particularly to a method of producing 3,4-diacyloxy-1-butene, with an excellent production efficiency, by separating 3,4-diacyloxy-1-butene from a reaction system.

2. Prior Art

There is known a method of producing 3,4-diacetoxy-1-butene, utilizing isomerization of 1,4-diacetoxy-2-butene.

Since isomerization of 1,4-diacetoxy-2-butene to 3,4-diacetoxy-1-butene is an equilibrium reaction, the isomerization is terminated when the ratio of isomers reaches equilibrium. Therefore, the yield rate of a desired isomerized product (i.e. 3,4-diacetoxy-1-butene) with respect to the supply amount of a starting material (i.e. 1,4-diacetoxy-2-butene) is generally low. Under the circumstances, various efficient production method are proposed.

For instance, JP10-212264A proposes a production method, wherein 1,4-diacyloxy-2-butene containing trans isomers of 90% or more is used as a starting material, based on the facts that 1,4-diacetoxy-2-butene contains trans isomers and cis isomers; and that isomerization of trans isomer proceeds rapidly in the presence of platinum chloride and/or chloroplatinate, whereas isomerization of cis isomer does not proceeds rapidly.

Specifically, 1,4-diacetoxy-2-butene containing trans isomers of 90% or more, and platinum chloride are supplied and heated to 120° C. (Example 1), or 100° C. (Example 2) in nitrogen atmosphere for isomerization. After the isomerization is terminated, 3,4-diacetoxy-1-butene is separated from the resulting mixture by a well-known method such as distillation.

Isomerization of 1,4-diacetoxy-2-butene to 3,4-diacetoxy-1-butene is usually terminated at an equilibrium state where the ratio of 3,4-diacetoxy-1-butene/1,4-diacetoxy-2-butene has reached about 40/60. Therefore, even if the invert rate of 1,4-diacetoxy-2-butene is increased by using 1,4-diacetoxy-2-butene containing a large amount of trans isomers, the isomerization is terminated when the reaction mixture reaches the aforementioned ratio of isomers. There is no case that the content rate of 3,4-diacetoxy-1-butene in the solution after the isomerization, i.e. the yield rate of 3,4-diacetoxy-1-butene with respect to the supply amount of the starting material, may exceed 40%. In the above method, the isomerized product (3,4-diacetoxy-1-butene) is separated from the resulting mixture by distillation or a like process, and 1,4-diacetoxy-2-butene (residues) is reused as the starting material. The reuse of the 1,4-diacetoxy-2-butene, however, requires separation of trans isomers and cis isomers. Under these circumstances, there is a demand for a further improvement on the production efficiency.

### SUMMARY OF THE INVENTION

An object of the invention is to provide a method for increasing the yield of a isomerized product per unit time by continuously producing the isomerized product, while suppressing the isomerization from attaining equilibrium and thereby avoiding termination of the isomerization.

The inventors have found that a reaction time for reaching an equilibrium can be extended by distilling away an isomerized product from a reaction system, and that the yield of the isomerized product per unit time can be increased by utilizing the fact that the boiling point of 3,4-diacyloxy-1-butene (desired isomerized product) is lower than the boiling point of 1,4-diacyloxy-2-butene (starting material), and accomplished the invention.

The present invention is a method of producing 3,4-diacyloxy-1-butene comprising isomerization by heating 1,4-diacyloxy-2-butene in the presence of an isomerization catalyst, wherein the isomerization is performed while distilling away 3,4-diacyloxy-1-butene using a reactor equipped with a distillation device.

In another aspect, this invention is a method of producing 3,4-diacyloxy-1-butene comprising steps of: continuously supplying 1,4-diacyloxy-2-butene to a reactor equipped with a distillation column connected to the upper portion thereof; isomerizing 1,4-diacyloxy-2-butene to 3,4-diacyloxy-1-butene in the presence of an isomerization catalyst at a temperature not less than the boiling point of 3,4-diacyloxy-1-butene; and continuously distilling away 3,4-diacyloxy-1-butene from the top of the distillation column.

In the method of the present invention, 3,4-diacyloxy-1-butene produced is distilled away during isomerization, thereby preventing the isomerization from attaining equilibrium. Accordingly, yield rate of 3,4-diacyloxy-1-butene with respect to the supply amount of the starting material (1,4-diacyloxy-1-butene) can be increased, regardless of the same invert rate of 1,4-diacyloxy-2-butene to 3,4-diacyloxy-1-butene.

### BRIEF DESCRIPTION OF THE DRAWING

FIG. 1 is a schematic diagram showing an embodiment of a reactor unit for use in a production method of the invention.

### DETAILED DESCRIPTION OF THE INVENTION

A method of producing 3,4-diacyloxy-1-butene of the invention comprises isomerizing 1,4-diacyloxy-2-butene through heating in the presence of an isomerization catalyst, wherein the isomerization is performed, while distilling away 3,4-diacyloxy-1-butene produced during the isomerization using a reactor equipped with a distillation device.

1,4-diacyloxy-2-butene is a compound expressed by the following general formula (1):

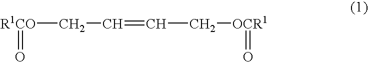

where R¹ is a saturated aliphatic hydrocarbon group. Examples of the saturated aliphatic hydrocarbon group include alkyl groups such as methyl, ethyl, propyl, isopropyl, butyl, t-butyl, amyl, and isoamyl; and cycloalkyl groups such as cyclopentyl and cyclohexyl. Among these, alkyl groups having 1 to 4 carbon atoms are preferred, and methyl is particularly preferred. A preferred example of 1,4-diacyloxy-2-butene is 1,4-diacetoxy-2-butene.

Both of cis isomers and trans isomers may be used as 1,4-diacyloxy-2-butene, but trans isomers may be preferably used. 1,4-diacyloxy-2-butene containing trans isomers of at least 90 wt % is particularly preferable. This is because trans isomers can be rapidly converted to 3,4-diacyloxy-1-butene, whereas cis isomers are hardly likely to be converted.

Any material containing 1,4-diacyloxy-2-butene as a primary ingredient may be used for a starting material. 1,4-diacyloxy-2-butene containing a certain amount of by-product in the production process of the 1,4-diacyloxy-2-butene, other than cis isomer, may be used as a starting material. For instance, 1,4-diacyloxy-2-butene containing 0.1 to 10 wt % (10 wt % excluded) of 3,4-diacyloxy-1-butene may be also used as the starting material.

3,4-diacyloxy-1-butene is a compound represented by the following formula (2), wherein the acyloxy group at the 1-position is transferred to the 3-position in 1,4-diacyloxy-2-butene, and a double bond is transferred to the 1-position by heating 1,4-diacyloxy-2-butene in the presence of an isomerization catalyst.

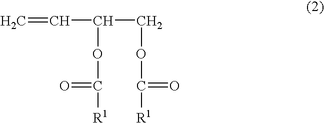

where R¹ is the same as R¹ in the formula (1).

For instance, in the case where 1,4-diacetoxy-2-butene is used as a starting material, the isomerized product is 3,4-diacetoxy-1-butene.

A well-known isomerization catalyst for use in converting 1,4-diacyloxy-2-butene to 3,4-diacyloxy-1-butene may be used. Examples of the isomerization catalyst are compounds of a metal selected from metals belonging to groups VIII to X in the periodic table.

Examples of the metal include transition metals such as iron, cobalt, nickel, ruthenium, rhodium, platinum, iridium, osmium, and palladium. Preferable examples of the metal include platinum metals such as ruthenium, rhodium, palladium, osmium, iridium, and platinum, and particularly preferred examples of the metal include metals belonging to group X in the periodic table, and more preferably, platinum. Examples of the metal compounds are acetate, acetyl acetoxynate, halide, sulfate, nitrate, amine compounds, pyridine compounds, alkene compounds, phosphine coordinate compounds, and phosphite coordinate compounds. Among these, platinum salts are preferably used. The form of the metal compounds is not specifically limited, and monomers, dimers, or polymers of the metal compounds may be used.

The amount of these catalysts charged for the isomerization is not specifically limited. However, the catalyst in the range of from 1×10⁻⁸ (0.01 molar ppm) to 1 molar equivalent, and preferably from 1×10⁻⁷ (0.1 molar ppm) to 0.001 molar equivalent, or from 1×10⁻⁶ to 0.0001 molar equivalent with respect to the amount of 1,4-diacyloxy-2-butene as a starting material may be used from the viewpoint of catalytic activity and economic efficiency.

It is preferred to use a catalyst additive with the isomerization catalyst. Well-known catalyst additives may be used, such as a compound coordinatable to a metal compound used as the catalyst. Examples of the catalyst additives include phosphorous compounds, nitrogen compounds, and allyl compounds. In the case where a compound of a metal selected from metals of groups VIII to X in the periodic table is used as the isomerization catalyst, an organic phosphorous compound is preferably used as the catalyst additive. Examples of the organic phosphorous compound are phosphines, phosphites, phosphonites, and phosphinates. These compounds may act as a monodentate or polydentate ligand, or a mixture thereof. The amount of the catalyst additive is e.g. normally in the range from 0.1 to 10 molar equivalents, and preferably from 1 to 4 molar equivalents with respect to the amount of the metal compound.

1,4-diacyloxy-2-butene and an isomerization catalyst are charged into a reactor, and preferably a catalyst additive is further charged. In addition, a solvent, a polymerization inhibitor, a defoamer, or the like may be charged.

The inventive method is preferably performed without a solvent, but may not exclude use of a solvent. The solvent usable for the method of the invention is not specifically limited, as far as the solvent is an organic solvent capable of dissolving a catalyst and a starting material compound, and having a boiling point higher than the heating temperature at a reaction pressure. Such solvent is not distilled away with a desired isomerized product. Examples of the solvent are carboxylic acids having 5 to 20 carbon atoms; alcohols having 10 to 20 carbon atoms; ethers such as diglime, diphenylether, dibenzylether, tetrahydrofurane, and dioxane; amides such as N-methylpyrrolidone, dimethylformamide, and dimethylacetoamide; ketones such as cyclohexanone; esters such as butyl acetate, γ-butylolactone, and di(n-octyl)phthalate; aromatic hydrocarbons such as toluene, xylene, and dodecylbenzene; and substances having a high boiling point and generated as a by-product in the reaction system.

The isomerization is performed with use of a reactor unit assembled by mounting a distillation device on a reactor. The distillation device may be provided with a reflux mechanism. The installation position of the distillation device is not specifically limited, but preferably, the distillation device may be integrally installed at an upper part of the reactor, in order to collect gas of a desired product. The distillation device may preferably be a distillation column.

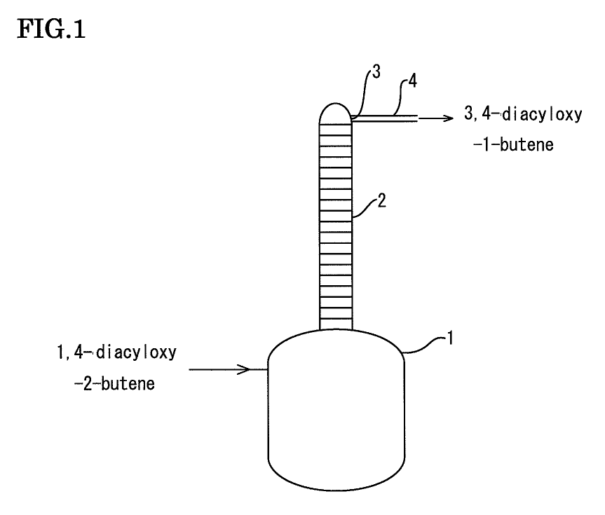

A specific preferred example of the reactor unit having a distillation device is shown in FIG. 1. In the reactor unit shown in FIG. 1, 1 indicates a reactor, and 2 indicates a distillation column. A distillation port 3 is formed in the vicinity of a top part of the distillation column 2 to distill away 3,4-diacyloxy-1-butene. A pressure reducing device such as a vacuum pump is provided at an appropriate position of a separation/collecting line 4 to distill away 3,4-diacyloxy-1-butene, and reduce the pressure in the reaction system. Preferably, the distillation column 2 may be provided with a certain number of column plates or filled with a column packing capable of theoretically separating 3,4-diacyloxy-1-butene from the other compounds (particularly, 1,4-diacyloxy-2-butene).

A starting material of the isomerization may be directly supplied to the reactor. When a reaction product is used as a starting material, the reaction product is introduced to the reactor for the isomerization. For instance, in the case where a product obtained by acyloxydation of butadiene is used as a starting material, an acyloxylated product may be introduced directly from a separation unit of the acyloxylated product to a port of the reactor for the isomerization.

The starting material may be supplied to the reactor batchwise, but preferably supplied continuously or semicontinuously. In the method of the invention, since 3,4-diacyloxy-1-butene being produced is distilled away, the starting material can be continuously supplied to a reactor, even using a constant volume reactor. The isomerization is allowed to continue by continuous supply of the starting material. As a result, the yield of the isomerized product per unit time can be increased.

The isomerization is performed while heating. The heating temperature may be properly selected depending on the starting material and/or the isomerized product, but preferably be a temperature equal to or higher than the boiling point (at a reaction pressure) of 3,4-diacyloxy-1-butene. Preferably, the isomerization is performed in a liquid phase. Also, the upper limit of the heating temperature is preferably lower than the boiling point (at a reaction pressure) of 1,4-diacetoxy-2-butene for separating 3,4-diacyloxy-1-butene from the reaction system by distillation. Alternatively, as far as a vapor stream of 1,4-diacetoxy-2-butene can be recovered to the reactor by reflux, the heating temperature may be equal to or higher than the boiling point of 1,4-diacetoxy-2-butene. Accordingly, the reaction temperature is normally in the range of from the boiling point (Tb_{(3,4)}) (at a reaction pressure) of 3,4-diacyloxy-1-butene to Tb_{(3,4)}+50° C., and preferably from Tb_{(3,4)} to Tb_{(3,4)}+30° C.

Both of the boiling points of the starting material and the isomerized product may depend on the reaction pressure, R¹ in the general formula, and the type of stereo isomers such as cis isomers or trans isomers. For instance, in the case where R¹ is methyl, the boiling point of 1,4-diacetoxy-2-butene is in the range from 222 to 230° C. at 1 atm, and the boiling point of 1,4-diacetoxy-2-butene is in the range from 120 to 127° C. at 18 mmHg (=about 0.02 atm). On the other hand, the boiling point of 3,4-diacetoxy-1-butene is in the range from about 206 to 208° C. at 1 atm, and the boiling point of 3,4-diacetoxy-1-butene is in the range from 95 to 96° C. at 10 mmHg (=about 0.01 atm).

The pressure in the reaction system is not specifically limited, but is preferably 1 atm or less to lower the boiling point of 3,4-diacetoxy-1-butene. Since boiling points are lowered under a reduced pressure, the heating temperature can be lowered by reduction of the pressure in the reaction system. Also, side reactions can be suppressed by lowering the heating temperature.

The air suction port for reducing the pressure in the reaction system is not specifically limited, but use of the distillation port of 3,4-diacyloxy-1-butene in common as the air suction port is preferred because the arrangement enables to reduce the pressure in the reaction system while distilling away the isomerized product from the reaction system. The installation positions of the air suction port and the distillation port are not specifically limited. However, if the installation position is close to the surface of the reaction solution, collecting the isomerized product may be insufficient. Therefore, the installation position is preferably an intermediate part or an upper part of the distillation device, more preferably the upper part, and particularly preferably a top part.

The temperature of the distillation device is lowered toward the top part thereof. In the case where 3,4-diacetoxy-1-butene is distilled away from the distillation device, it is preferable to set the temperature at the distillation port substantially equal to the boiling point of 3,4-diacyloxy-1-butene. If the temperature at the distillation port is lower than the boiling point of 3,4-diacyloxy-1-butene, 3,4-diacyloxy-1-butene may be liquefied at a site closer to the reactor than the distillation port, and the liquefied 3,4-diacyloxy-1-butene may be refluxed to the reactor. For this reason, it is preferable to set the temperature of the distillation port to at least a temperature capable of separating the desired isomerized product from the reaction mixture.

As described above, the isomerization can be performed while distilling away 3,4-diacyloxy-1-butene. In particular, use of the reactor unit assembled by integrally mounting the distillation device on the top part of the reactor enables to distill away 3,4-diacyloxy-1-butene during the isomerization. This is advantageous in collecting 3,4-diacyloxy-1-butene of a high purity.

According to the inventive method, since the interior of the reactor is heated to a temperature equal or higher than the boiling point of the desired isomerized product, generated vapors are drawn into the distillation device. The vapors to be drawn into the distillation device may contain 1,4-diacyloxy-2-butene gas, and gas of impurities, in addition to 3,4-diacyloxy-1-butene gas. However, compounds (particularly, 1,4-diacyloxy-2-butene) which have a boiling point higher than the boiling point of 3,4-diacyloxy-1-butene are liquefied and refluxed to the reactor, while gas of 3,4-diacyloxy-1-butene can be guided upward to a position near the distillation port. Thus, 3-4-diacyloxy-1-butene is distilled away through the distillation port, followed by cooling, whereby 3,4-diacyloxy-1-butene (liquid) can be obtained. In the case where 3,4-diacyloxy-1-butene is already liquefied at a position near the distillation port, 3,4-diacyloxy-1-butene (liquid) may be distilled away from the reaction system by providing column plates and guiding the liquid into the distillation port along the column plates.

Isomerization of 1,4-diacyloxy-2-butene to 3,4-diacyloxy-1-butene is an equilibrium reaction. In the case where isomerization of 1,4-diacetoxy-2-butene to 3,4-diacetoxy-1-butene is carried out at 150° C., the isomerization attains equilibrium where a mixture comprising about 60 to 65 mol % of 1,4-diacetoxy-2-butene and about 35 to 40 mol % of 3,4-diacetoxy-1-butene is generated. However, continuously distilling away 3,4-diacetoxy-1-butene from the reaction system as described above enables to avoid attaining the equilibrium, thereby suppressing termination of the isomerization. Also, the above method enables to keep the rate of generating the isomerized product high, thereby enabling to increase the yield (or the yield rate) of the isomerized product with respect to the supply amount of the starting material to the reaction system.

According to the producing method of the present invention, since 1,4-diacetoxy-2-butene can be supplied continuously to the reaction system by continuous distillation away of 3,4-diacetoxy-1-butene, termination of the isomerization caused by luck of the starting material can be avoided. Accordingly, the inventive method enables to increase the yield of the reaction product per unit time, as compared with a conventional method employing a batch process comprising: supplying a predetermined amount of a starting material to a reactor; taking out the obtained mixture from the reactor after the isomerization is terminated; and distilling. Also, the inventive method suppresses the isomerization from attaining equilibrium. This enables to keep the rate of generating the isomerized product high, thereby enabling to increase the yield rate of the isomerized product with respect to the supply amount of the starting material to the reaction system.

In view of the above, in the case where 3,4-diacetoxy-1-butene is produced from 1,4-diacetoxy-2-butene, for instance, the temperature in the reactor is normally kept in the range of from 100 to 235° C., and preferably from 120 to 160° C. depending on the magnitude of the pressure in the reactor. This is because an unduly high temperature may cause a side reaction. On the other hand, an unduly low reaction temperature may reduce the reaction rate, and make it difficult to distill away 3,4-diacetoxy-1-butene.

Also, the temperature at the distillation port, which 3,4-diacetoxy-1-butene passes through, is set lower than the temperature of the reactor e.g. normally in the range from 60 to 210° C., preferably from 80 to 180° C., and particularly preferably from 100 to 140° C.

Further, the pressure in the reaction system may be properly selected in the range from 1 to 120,000 Pa, but may be preferably selected in the range from 1 to 60,000 Pa, and more preferably from 1 to 15,000 Pa.

The shape of the distillation device to be mounted on the reactor is not specifically limited, but preferably be a column shape. Particularly, it is preferable to use a distillation column having a theoretically plate number of 5 to 50 for theoretically separating 3,4-diacetoxy-1-butene (isomerized product) from 1,4-diacetoxy-2-butene (starting material). This is because an unduly small plate number may make separation of 3,4-diacetoxy-1-butene and 1,4-diacetoxy-2-butene insufficient.

In the inventive method, performing distillation simultaneously with isomerization enables to prevent the isomerization from attaining equilibrium, thereby enabling to suppress termination of the isomerization. Also, since 1,4-diacetoxy-2-butene can be continuously supplied, the yield rate of 3,4-diacetoxy-1-butene with respect to the supply amount of the starting material, or the yield of 3,4-diacetoxy-1-butene per unit time can be increased while allowing use of the conventional starting material and/or catalyst. This is advantageous in enhancing the production efficiency.

### EXAMPLE

As a starting material, 700 weight parts of 1,4-diacetoxy-2-butene (93.5 wt % of trans-1,4-diacetoxy-2-butene, 2.0 wt % of cis-1,4-diacetoxy-2-butene, 3.6 wt % of 3,4-diacetoxy-1-butene, and 0.9 wt % of other ingredients) was supplied to a 2000-weight part volume reactor communicated with a continuous older-shaw fractional distillation column (32φ, 40 column plates) equipped with a rotating stirrer therein. Nitrogen gas was blown into the reactor for 1 hour to release oxygen from the reactor. Thereafter, 0.2 weight parts of platinum chloride (II), and 0.2 weight parts of triphenylphosphine (each corresponding to 0.0002 mol with respect to 1 mol of trans-1,4-diacetoxy-2-butene) were supplied to the reactor, and the mixture was heated up to 160° C. while being stirred. A total reflux was conducted for 3 hours after the reaction vapors started to pass through the top part of the distillation column. The pressure in the reaction system was set to 13,300 Pa (=99 mmHg=0.1 atm) by sucking the top part of the distillation column by a vacuum pump. The boiling point of 3,4-diacetoxy-1-butene was 134° C., the boiling point of trans-1,4-diacetoxy-2-butene was 168° C., and the boiling point of cis-1,4-diacetoxy-2-butene was 163° C. all at 0.1 atm. Accordingly, the reaction temperature was higher than the boiling point of 3,4-diacetoxy-1-butene by 26° C., and lower than the boiling points of cis- and trans-1,4-diacetoxy-2-butene.

Thereafter, the starting material containing 1,4-diacetoxy-2-butene was continuously supplied in the amount of 40 weight parts/hour, while distilling away 3,4-diacetoxy-1-butene through the top part of the distillation column in the amount of 40 weight parts/hour. The isomerization was performed for 12 hours.

The yield of 3,4-diacetoxy-1-butene as a resulting product was 443 parts by weight, and the yield rate thereof with respect to the supply amount of the starting material was 37.5%. Analysis of gas chromatography of the resulting product showed that the following: 98 wt % of 3,4-diacetoxy-1-butene, 0.08 wt % of cis-1,4-diacetoxy-2-butene, 0.09 wt % of trans-1,4-diacetoxy-2-butene, and 1.83 wt % of impurities.

### COMPARATIVE EXAMPLE

A reactor unit having 1000-weight part volume vessel equipped with a rotating stirrer therein was used. 1,4-diacetoxy-2-butene as one employed in Example was employed. 350 weight parts of the 1,4-diacetoxy-2-butene was supplied batchwise to the reactor. Further, 0.02 weight parts of platinum chloride (II), and 0.02 weight parts of triphenylphosphine (each corresponding to 0.00004 mol with respect to 1 mol of 1,4-diacetoxy-2-butene) were supplied as catalysts to the reactor. Nitrogen gas was blown into the reactor to release oxygen from the reactor. Thereafter, the mixture was heated up to 120° C. while being stirred. The isomerization was performed for 6.5 hours (6 hours excluding the time required for temperature raising). Analysis of the obtained product by gas chromatography showed that the invert rate was 20%. The temperature was then raised to 150° C., and the isomerization was still performed for 8 hours. As a result of the further isomerization, the invert rate has increased to 36.5%.

After the isomerization was terminated, the obtained mixture was put in another distillation column (32φ, 40 column plates). Then, the temperature in the vessel was raised to 160° C., and a total reflux was conducted for 3 hours after the generated vapors started to pass through the top part of the distillation column. The pressure in the reaction system was set to 13,300 Pa (=99 mmHg=0.1 atm) by sucking the top part of the distillation column by a vacuum pump.

The yield of the isomerized product is 115 parts by weight. The yield rate of 3,4-diacetoxy-1-butene with respect to the supply amount of the starting material was 32.9%.

The results on Example and Comparative Example are shown in Table 1.

TABLE 1

| | | Comparative |
|---|---|---|
| | Example | Example |
| Conditions of isomerization | 0.1 atm | Atmospheric |
| | 160° C. | pressure |
| | | 120° C. → |
| | | 150° C. |
| Supply amount (weight part) | 700 + 40 × 12 = | 350 |
| | 1180 | |
| Reaction time (hour) | 12 | 14.5 |
| Yield of the desired product | 443 | 115 |
| (weight part) | | |
| Yield rate relative to supply amount | 37.5 | 32.9 |
| (%) | | |
| Yield rate of the desired product | 3.13 | 2.27 |
| per unit time (%/h) | | |
| (Yield rate relative to supply | | |
| amount/reaction time) | | |

As is obvious from Table 1, comparison of the results of Example and Comparison Example illustrates that, even using the same starting material, the yield rate of the isomerized product with respect to the supply amount of the starting material was higher in Example. It is conceived that distilling away 3,4-diacetoxy-1-butene from the reaction system enables to prevent the reaction from attaining equilibrium, thereby promoting the isomerization. Also, since the starting material is continuously supplied in Example, the yield of the isomerized product per unit time was increased. The above result shows that the method of Example is advantageous in enhancing the production efficiency, as compared with the conventional method employing a batch process.

What is claimed is:

1. A method of producing 3,4-diacyloxy-1-butene by isomerization by heating 1,4-diacyloxy-2-butene in the presence of an isomerization catalyst, wherein the isomerization is performed while distilling away 3,4-diacyloxy-1-butene using a reactor equipped with a distillation device.

2. A method according to claim 1, wherein the isomerization is performed at a temperature of not less than a boiling point of 3,4-diacyloxy-1-butene.

3. A method according to claim 2, wherein the isomerization is performed at a temperature in the range from the boiling point of 3,4-diacyloxy-1-butene to a temperature of 50° C. higher than the boiling point thereof.

4. A method according to claim 3, wherein the isomerization is performed in the range from 100 to 235° C.

5. A method according to claim 1, wherein the isomerization is performed under a reduced pressure.

6. A method according to claim 5, wherein the isomerization is performed under the reduced pressure in the range from 1 to 120000 Pa.

7. A method according to claim 1, wherein the isomerization catalyst is a compound of a metal selected from metals of group VIII to group X in the periodic table.

8. A method according to claim 1, wherein an amount of the isomerization catalyst is in the range of 1×10⁻⁸ to 1 molar equivalent with respect to an amount of 1,4-diacyloxy-2-butene supplied as a starting material.

9. A method according claim 1, wherein the isomerization is performed without a solvent.

10. A method according to claim 1, wherein the 1,4-diacyloxy-2-butene contains 3,4-diacyloxy-1-butene in a concentration of 0.1 to 10 (10 excluded) % by weight.

11. A method according to claim 1, wherein the reactor equipped with a distillation device is a reactor equipped with a distillation column connected to the upper portion thereof.

12. A method according to claim 11, wherein the isomerization is performed under a condition of continuously supplying 1,4-diacyloxy-2-butene to the reactor and of continuously collecting 3,4-diacyloxy-1-butene from the top of the distillation column.

13. A method according to claim 12, wherein the temperature at the top of the distillation column falls in the range from 60 to 120° C.

14. A method according to claim 1, wherein the 1,4-diacyloxy-2-butene is a compound represented by the general formula (1), and the 3,4-diacyloxy-1-butene is a compound represented by the general formula (2): 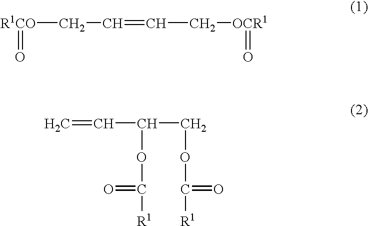
where R¹ is an alkyl group having 1 to 4 carbon atoms.

15. A method according to claim 14, wherein the 1,4-diacyloxy-2-butene is 1,4-diacetoxy-2-butene, and the 3,4-diacyloxy-1-butene is 3,4-diacetoxy-1-butene.

16. A method of producing 3,4-diacyloxy-1-butene comprising steps of:
continuously supplying 1,4-diacyloxy-2-butene to a reactor equipped with a distillation column connected to the upper portion thereof;
isomerizing 1,4-diacyloxy-2-butene to 3,4-diacyloxy-1-butene in the presence of an isomerization catalyst at a temperature not less than a boiling point of 3,4-diacyloxy-1-butene; and
continuously distilling away 3,4-diacyloxy-1-butene from the top of the distillation column.

17. A method according to claim 16, wherein inside of the reactor is under a reduced pressure.

18. A method according to claim 16, wherein the isomerization is performed without a solvent.

19. A method according to claim 16, wherein the temperature at the top of the distillation column falls in the range from 60 to 120° C.
